(19) United States
(12) Patent Application Publication
(10) Pub. No.: US 2009/0264814 A1
(43) Pub. Date: Oct. 22, 2009

(54) DEVICE FOR MEDICAL TREATMENT DECISION SUPPORT AND/OR MONITORING THE STATUS OF A PATIENT

(75) Inventors: Hendrika Cecilia Krijnsen, Eindhoven (NL); Geert Langereis, Eindhoven (NL); Michel Paul Barbara Van Bruggen, Eindhoven (NL); Ventzeslav Petrov Iordanov, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/375,743

(22) PCT Filed: Jul. 17, 2007
(86) PCT No.: PCT/IB2007/052839

§ 371 Date: Jan. 30, 2009

(30) Foreign Application Priority Data
Aug. 2, 2006 (EP) 06118300.0

(51) Int. Cl. A61M 31/00; A61B 5/00

(52) U.S. Cl. 604/66; 600/300

(57) ABSTRACT

The invention relates to a device for medical treatment decision support and/or monitoring the status of a patient, the device comprising a first measuring means at the ipsilateral side and a second measuring means at the contralateral side and a comparison means in order to find the optimal and/or suboptimal time for drug treatment and/or other medical treatment steps.

This invention is in the field of devices for the analysis of a patient as well as devices for therapeutic administration, especially for cancer treatment, more especially for breast cancer treatment. The concept of homeostasis stipulates that there is constancy of the endogenous compounds in blood. This is a most powerful construct in biology, and has influenced not only the teaching and understanding of medical science but also the practice of clinical medicine. According to this concept, the risk of the occurrence and exacerbation of disease is independent of the time of day, day of month, and month of year, as is the response of patients to diagnostic tests and medications. However, most biological functions and processes are anything but constant; Findings from the field of biologic rhythm study (chronobiology) challenge the concept of homeostasis, as well as many of the assumptions and procedures of clinical medicine.

Many biological functions wax and wane in cycles that repeat on a daily, monthly or annual basis. Such patterns do not reflect simply an organism's passive response to environmental changes, such as daily cycles of light and darkness. Rather, they reflect the organism's biological rhythms, that is, its ability to keep track of time and to direct changes in function accordingly.

Especially in the field of cancer treatment the concept of taking into account circadian fluctuations and/or circadian circles has become more prominent in recent times.

In 1992 it was discovered by Hori et al, Circadian variation of tumor blood flow in rat subcutaneous tumors and its alteration by angiotensin II-induced hypertension, Cancer Research, Vol. 52, pp. 912-916 (1992) that circadian fluctuations exist in tissue blood flow of rat tumors. In 1995 it was found that the time during which tumor tissue blood flow increases coincides with the time during which tumor growth becomes more rapid.

This supports the idea that there may be an optimal time at which the anti-cancer drugs have highest treatment efficacy. Commonly used anti-cancer drugs effects are believed to be highest when cancer cells are actively dividing, something that in most concepts in the field corresponds with highest tumor blood flow.

However, in prior art, e.g. in Simpson, H. W., Sir James Young Simpson memorial lecture 1995, J R Coll Surg Edinb, Vol. 41, pp. 359-370 (1996) rhythms were measured by a daily measurement of a body parameter (e.g. temperature) at a constant time during the day, e.g. 8 o'clock in the evening. This once-a-day measurement provides a snap-shot of the rhythm of a tumor, only providing information on the infradian rhythm where ultradian rhythms may also be present. The ultradian rhythms are not properly identified when only 1 measurement (or a series of measurements in a short time interval) is being performed every 24 h.

It is therefore an object of the present invention to provide a device for the analysis and/or drug administration, especially in cancer treatment, which is adapted to take into account the rhythm of a patient.

This object is achieved by a device according to claim 1 of the present invention. Accordingly, a device for medical treatment decision support and/or monitoring the status of a patient is provided, comprising
a) a first measuring means, which measures at least one first body parameter at the ipsilateral side of a tumor for at least ≧1 measuring cycle,
b) a second measuring means which measures at least one second body parameter at the contralateral side of a tumor for at least ≧1 measuring cycle,
c) a comparison means to compare the data of the first and second measuring means and/or the fitting curves of derived from the first and second measuring means.

The term “ipsilateral” and/or “ipsilateral side” especially means and/or includes on or relating to the same side (of the body), i.e. near or at the diseased or cancerous tissue.

The term “contralateral” and/or “contralateral side” especially means and/or includes on or related to the side opposite to the injured/diseased/cancerous tissue.

By doing so, at least one of the following advantages is achieved for most of the applications within the present invention:
For a wide range of applications within the present invention, no initial longer term measurements are needed that have to be analysed before the therapy can start. This allows a wide range of applications within the present invention to speed-up the process of therapy providing an earlier start, which is desired in fast-growing and advanced stage tumors
For a wide range of applications within the present invention, fewer side effects can be noticed.
For a wide range of applications within the present invention the therapeutic dose (such as radiation, drugs . . . ) may be increased to increase treatment. efficacy.
For a wide range of applications within the present invention, also a higher efficacy of treatment can be achieved.
For a wide range of applications within the present invention, a better efficiency is noticed in a case of a temporary refusal of the therapy by the patient. In the mean time, rhythm may have changed, which is taken into account by the present invention.
For a wide range of applications within the present invention, more information on the actual status of the tumor side and thus on the tumor doubling time/growth rate of the tumor (stage) can be gathered.
For a wide range of applications within the present invention, more information on the effectiveness of the therapy at the tumor side is feasible, which may lead to a more effective treatment, which results in a change in amplitude of the rhythm and the acrophase.
For a wide range of applications within the present invention, the invention allows the observation of desynchronization. This observation of desynchronization would be lost in studies relying solely on cosinor treatment restricted to the fit of a single period assumed to be equal to 24 hrs.
For a wide range of applications within the present invention, the invention allows better treatment especially for fast growing tumors (tumor doubling times are short, metabolic heat production is high). For a wide range of applications within the present invention, also a higher efficacy of treatment can be achieved.
For a wide range of applications within the present invention, the invention allows estimation of rhythms other than 24 hrs.
It may allow better timing of therapy even within the general operating hours of a hospital.

The term “measuring cycle” means and/or includes especially that a body parameter of the patient is measured, which is known and/or believed to behave in a cyclic and/or periodic manner, e.g. the body temperature.

According to an embodiment of the present invention, the at least one first body parameter includes body temperature, core body temperature, skin surface temperature, activity (body or brain), heart rate, melatonin level, triacylglycerol level, cortisol level, blood pressure, interstitial fluid pressure.

According to an embodiment of the present invention, the length of the measuring cycle(s) is from ≧7 h to ≦48 h. This has been shown to be sufficient in practice for a wide range of applications within the present invention. According to an embodiment of the present invention, the length of the measuring cycle(s) is from ≧8 h to ≦30 h.

According to an embodiment of the present invention, the number and nature of the second body parameter(s) are identical to the first body parameter(s).

According to an embodiment of the present invention the number and/or nature of the second body parameter(s) differ to those of to the first body parameter(s).

In the latter case, it is especially preferred for a wide range of applications to use normalized data and/or data derived from fitting curves as will be described later on.

According to an embodiment of the present invention, the device comprises a fitting means which generates at least one first fitting curve from the data of the first measuring means to determine the acrophase, amplitude, mesor and/or period and/or a fitting means which generates at least one second fitting curve from the data of the second measuring means to determine the acrophase, amplitude, mesor and/or period.

Such a fitting has been shown in practice to enhance the predictability of the behavior of the first and/or second body parameters for a wide range of applications within the present invention, which may help to increase the performance of the device according to the invention.

According to an embodiment of the present invention, the device comprises a normalizing means for normalizing the data from the comparison means in order to normalize either the first and/or second body parameter data and/or the difference of the first and second fitting curve and/or data.

The term “normalizing” means includes especially that from the data derived from the circadian curve, the normalizing curve is calculated by the equation:

Z=(X−mean(X))/standard deviation*100%

with X (also written as Xₜ) being the body parameter and mean(X)

being the mathematical average of Xₜ over a defined period. It should be noticed that usually X may have both positive and negative values.

The normalizing data obtained from the method used here are in % on a normalized scale; however it goes without saying that this is merely for the sake of better understanding and any person skilled in the art may easily transform the data to any given scale known in the field.

It has been shown in a range of applications that such a normalizing step may be of use for the reason that the normalization of the difference between ipsilateral and contralateral side may be of value to provide changes in normalized data that may be used to start and/or stop a therapy, either drug therapy, hyperthermia or radiotherapy.

According to an embodiment of the present invention, the comparison means includes a prediction means which predicts the peak(s) in the difference between the data of the first and second measuring means and/or the fitting curves derived from the first and second measuring means especially for determining an optimal and/or suboptimal time for drug treatment and/or other medical treatment steps.

According to an embodiment of the present invention, the device comprises a drug administering device which comprises a drug release means, which starts a drug release program based upon the comparison means.

According to an embodiment of the present invention, the device comprises a radiotherapy device, which starts a radiotherapy program based upon the comparison means.

According to an embodiment of the present invention, the device comprises a hyperthermia device, which starts a hyperthermia program based upon the comparison means.

The term “based upon the comparison means” means and/or includes especially that upon the data derived from the comparison means certain start and/or stop signals are issued upon which a drug release and/or radiotherapy program is started and/or halted.

According to an embodiment of the present invention, the drug release program includes a delay of ≧0 and ≦24 hours prior to the release of drugs.

According to an embodiment of the present invention, the device comprises a drug administering device which is chosen from transdermal patches, epills, implants, minipumps, port-a-caths, or drug administering and/or releasing implants.

It should be noted that according to an embodiment of the present invention, the measuring, selection and/or curve generation and/or normalizing means are included in the drug administering and/or radiotherapy device, whereas according to another embodiment of the present invention, they are separate. In the latter case, according to an embodiment of the present invention, the data and/or a start signal are transferred to the drug administering device and/or radiotherapy device in order to start the drug release program when needed.

The present invention also relates to a method for the controlled release of drugs and/or monitoring the status of a patient, the method comprising the steps of
a) measuring at least one first body parameter of the patient for at least one first body parameter at the ipsilateral side of a tumor for at least ≧1 measuring cycle,
b) measuring at least one second body parameter of the patient for at least one second body parameter at the contralateral side of a tumor for at least ≧1 measuring cycle,
c) comparing the data out of the first and second body parameters in order to optionally start a drug release program and/or determine an optimal and/or suboptimal time for drug treatment and/or other medical treatment steps.

The invention furthermore relates to the use of a device for medical treatment and/or monitoring the status of a patient for the diagnosis and/or treatment of cancer, especially breast cancer.

It has been shown for a wide range of applications that a device according to the present invention may therefore take a greater account for these rhythms and therefore may be of use for the diagnosis and/or treatment of cancer, especially breast cancer.

A device according to the present invention may be of use in a broad variety of systems and/or applications, among them one or more of the following:
medical devices for the administering of drugs, for hyperthermia and for radiotherapy,
medical devices for treatment of chronic diseases.

The aforementioned components, as well as the claimed components and the components to be used in accordance with the invention in the described embodiments, are not subject to any special exceptions with respect to their size, shape, material selection and technical concept, so that the selection criteria known in the pertinent field can be applied without limitations.

Additional details, features, characteristics and advantages of the object of the invention are disclosed in the dependent claims, the figures and the following description of the respective figures, tables and examples.

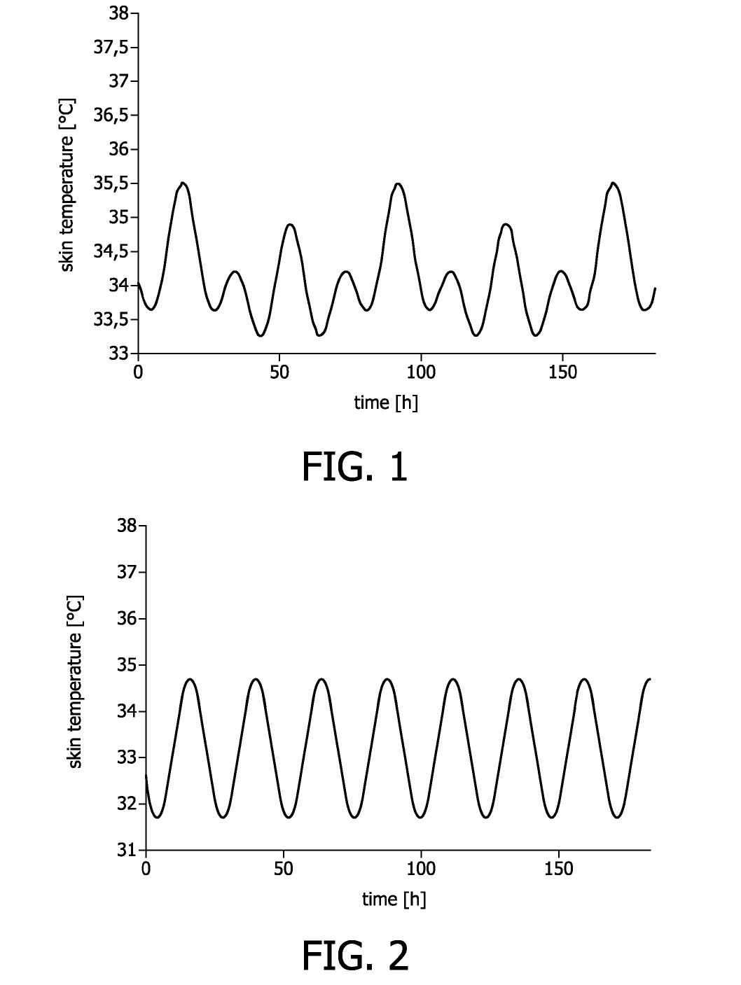

FIG. 1 shows a diagram of temperature plotted against time of the ipsilateral side of a tumor;

FIG. 2 shows a diagram of temperature plotted against time of the contralateral side of a tumor;

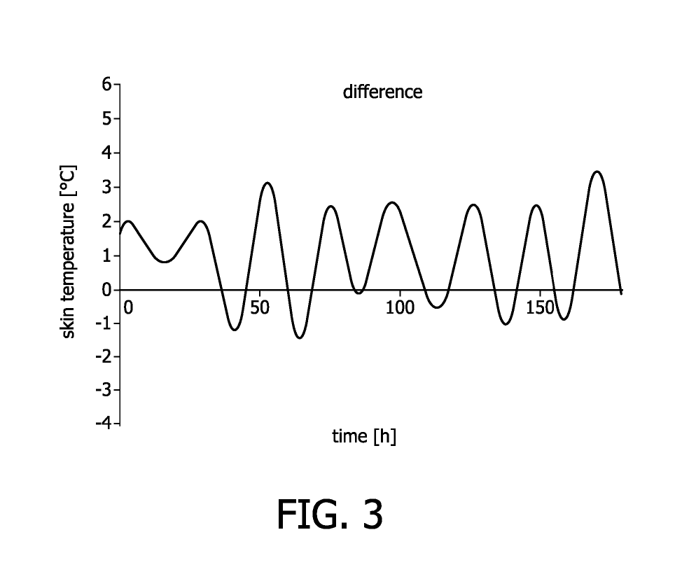

FIG. 3 shows a diagram of the difference in temperatures of the diagrams of FIGS. 1 and 2, and

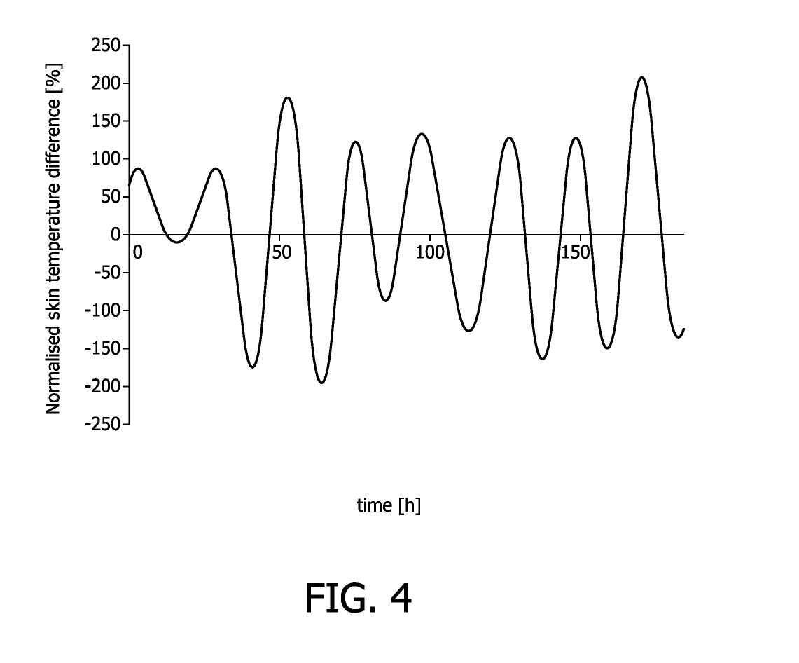

FIG. 4 shows a diagram of the difference in normalized temperatures of the diagrams of FIGS. 1 and 2.

The invention will furthermore be better understood with the following examples for some applications in which a device according to the present invention may be of use, but which are merely to be understood as exemplarily and not limiting for the present invention.

FIGS. 1 and 2 refer to the measurement of superficial temperature (ipsilateral and contralateral) of a tumor, whereby the temperature was measured over a period of 194 hrs by the hour.

The exact data are shown in Table I:

TABLE I

| time | ipsilateral | contralateral | |
|---|---|---|---|
| (hrs.) | temperature | temperature | difference |
| 0 | 34.06208 | 32.45 | 1.612078 |
| 1 | 33.95013 | 32.13934 | 1.810791 |
| 2 | 33.83232 | 31.90096 | 1.931358 |
| 3 | 33.72829 | 31.75111 | 1.977182 |
| 4 | 33.65687 | 31.7 | 1.956868 |
| 5 | 33.63384 | 31.75111 | 1.882726 |
| 6 | 33.67008 | 31.90096 | 1.769116 |
| 7 | 33.77019 | 32.13934 | 1.63085 |
| 8 | 33.9318 | 32.45 | 1.481801 |
| 9 | 34.14563 | 32.81177 | 1.333857 |
| 10 | 34.3963 | 33.2 | 1.196299 |
| 11 | 34.66383 | 33.58823 | 1.075597 |
| 12 | 34.92561 | 33.95 | 0.975607 |
| 13 | 35.15873 | 34.26066 | 0.898066 |
| 14 | 35.3423 | 34.49904 | 0.843263 |
| 15 | 35.45965 | 34.64889 | 0.810758 |
| 16 | 35.5 | 34.7 | 0.8 |
| 17 | 35.45965 | 34.64889 | 0.810758 |
| 18 | 35.3423 | 34.49904 | 0.843263 |
| 19 | 35.15873 | 34.26066 | 0.898066 |
| 20 | 34.92561 | 33.95 | 0.975607 |
| 21 | 34.66383 | 33.58823 | 1.075597 |
| 22 | 34.3963 | 33.2 | 1.196299 |
| 23 | 34.14563 | 32.81177 | 1.333857 |
| 24 | 33.9318 | 32.45 | 1.481801 |
| 25 | 33.77019 | 32.13934 | 1.63085 |
| 26 | 33.67008 | 31.90096 | 1.769116 |
| 27 | 33.63384 | 31.75111 | 1.882726 |
| 28 | 33.65687 | 31.7 | 1.956868 |
| 29 | 33.72829 | 31.75111 | 1.977182 |
| 30 | 33.83232 | 31.90096 | 1.931358 |
| 31 | 33.95013 | 32.13934 | 1.810791 |
| 32 | 34.06208 | 32.45 | 1.612078 |
| 33 | 34.14995 | 32.81177 | 1.338183 |
| 34 | 34.19908 | 33.2 | 0.999083 |
| 35 | 34.2 | 33.58823 | 0.611771 |
| 36 | 34.14954 | 33.95 | 0.199536 |
| 37 | 34.0512 | 34.26066 | −0.20946 |
| 38 | 33.91479 | 34.49904 | −0.58425 |
| 39 | 33.75531 | 34.64889 | −0.89358 |
| 40 | 33.5913 | 34.7 | −1.1087 |
| 41 | 33.44272 | 34.64889 | −1.20616 |
| 42 | 33.3287 | 34.49904 | −1.17034 |
| 43 | 33.26531 | 34.26066 | −0.99535 |
| 44 | 33.26371 | 33.95 | −0.68629 |
| 45 | 33.32876 | 33.58823 | −0.25947 |
| 46 | 33.45832 | 33.2 | 0.258316 |
| 47 | 33.64333 | 32.81177 | 0.831557 |
| 48 | 33.86861 | 32.45 | 1.418615 |
| 49 | 34.11436 | 32.13934 | 1.975021 |
| 50 | 34.35812 | 31.90096 | 2.457154 |
| 51 | 34.57709 | 31.75111 | 2.825974 |
| 52 | 34.75048 | 31.7 | 3.050485 |
| 53 | 34.8617 | 31.75111 | 3.110584 |
| 54 | 34.9 | 31.90096 | 2.999038 |
| 55 | 34.8617 | 32.13934 | 2.722356 |
| 56 | 34.75048 | 32.45 | 2.300485 |
| 57 | 34.57709 | 32.81177 | 1.765314 |
| 58 | 34.35812 | 33.2 | 1.158116 |
| 59 | 34.11436 | 33.58823 | 0.526133 |
| 60 | 33.86861 | 33.95 | −0.08139 |
| 61 | 33.64333 | 34.26066 | −0.61733 |
| 62 | 33.45832 | 34.49904 | −1.04072 |
| 63 | 33.32876 | 34.64889 | −1.32013 |
| 64 | 33.26371 | 34.7 | −1.43629 |
| 65 | 33.26531 | 34.64889 | −1.38358 |
| 66 | 33.3287 | 34.49904 | −1.17034 |
| 67 | 33.44272 | 34.26066 | −0.81794 |
| 68 | 33.5913 | 33.95 | −0.3587 |
| 69 | 33.75531 | 33.58823 | 0.167083 |
| 70 | 33.91479 | 33.2 | 0.714786 |
| 71 | 34.0512 | 32.81177 | 1.239426 |
| 72 | 34.14954 | 32.45 | 1.699536 |
| 73 | 34.2 | 32.13934 | 2.06066 |
| 74 | 34.19908 | 31.90096 | 2.298122 |
| 75 | 34.14995 | 31.75111 | 2.398843 |
| 76 | 34.06208 | 31.7 | 2.362078 |
| 77 | 33.95013 | 31.75111 | 2.19902 |
| 78 | 33.83232 | 31.90096 | 1.931358 |
| 79 | 33.72829 | 32.13934 | 1.588953 |
| 80 | 33.65687 | 32.45 | 1.206868 |
| 81 | 33.63384 | 32.81177 | 0.822065 |
| 82 | 33.67008 | 33.2 | 0.470078 |
| 83 | 33.77019 | 33.58823 | 0.181961 |
| 84 | 33.9318 | 33.95 | −0.0182 |
| 85 | 34.14563 | 34.26066 | −0.11503 |
| 86 | 34.3963 | 34.49904 | −0.10274 |
| 87 | 34.66383 | 34.64889 | 0.014936 |
| 88 | 34.92561 | 34.7 | 0.225607 |
| 89 | 35.15873 | 34.64889 | 0.509837 |
| 90 | 35.3423 | 34.49904 | 0.843263 |
| 91 | 35.45965 | 34.26066 | 1.198986 |
| 92 | 35.5 | 33.95 | 1.55 |
| 93 | 35.45965 | 33.58823 | 1.871418 |
| 94 | 35.3423 | 33.2 | 2.142301 |
| 95 | 35.15873 | 32.81177 | 2.346954 |
| 96 | 34.92561 | 32.45 | 2.475607 |
| 97 | 34.66383 | 32.13934 | 2.524485 |
| 98 | 34.3963 | 31.90096 | 2.495337 |
| 99 | 34.14563 | 31.75111 | 2.394517 |
| 100 | 33.9318 | 31.7 | 2.231801 |
| 101 | 33.77019 | 31.75111 | 2.019079 |
| 102 | 33.67008 | 31.90096 | 1.769116 |
| 103 | 33.63384 | 32.13934 | 1.494497 |
| 104 | 33.65687 | 32.45 | 1.206868 |
| 105 | 33.72829 | 32.81177 | 0.916521 |
| 106 | 33.83232 | 33.2 | 0.63232 |
| 107 | 33.95013 | 33.58823 | 0.361902 |
| 108 | 34.06208 | 33.95 | 0.112078 |
| 109 | 34.14995 | 34.26066 | −0.11071 |
| 110 | 34.19908 | 34.49904 | −0.29995 |
| 111 | 34.2 | 34.64889 | −0.44889 |
| 112 | 34.14954 | 34.7 | −0.55046 |
| 113 | 34.0512 | 34.64889 | −0.59769 |
| 114 | 33.91479 | 34.49904 | −0.58425 |
| 115 | 33.75531 | 34.26066 | −0.50535 |
| 116 | 33.5913 | 33.95 | −0.3587 |
| 117 | 33.44272 | 33.58823 | −0.1455 |
| 118 | 33.3287 | 33.2 | 0.128699 |
| 119 | 33.26531 | 32.81177 | 0.453538 |
| 120 | 33.26371 | 32.45 | 0.813709 |
| 121 | 33.32876 | 32.13934 | 1.189416 |
| 122 | 33.45832 | 31.90096 | 1.557354 |
| 123 | 33.64333 | 31.75111 | 1.892218 |
| 124 | 33.86861 | 31.7 | 2.168615 |
| 125 | 34.11436 | 31.75111 | 2.36325 |
| 126 | 34.35812 | 31.90096 | 2.457154 |
| 127 | 34.57709 | 32.13934 | 2.437746 |
| 128 | 34.75048 | 32.45 | 2.300485 |
| 129 | 34.8617 | 32.81177 | 2.049924 |
| 130 | 34.9 | 33.2 | 1.7 |
| 131 | 34.8617 | 33.58823 | 1.273467 |
| 132 | 34.75048 | 33.95 | 0.800485 |
| 133 | 34.57709 | 34.26066 | 0.316426 |
| 134 | 34.35812 | 34.49904 | −0.14092 |
| 135 | 34.11436 | 34.64889 | −0.53453 |
| 136 | 33.86861 | 34.7 | −0.83139 |
| 137 | 33.64333 | 34.64889 | −1.00556 |
| 138 | 33.45832 | 34.49904 | −1.04072 |
| 139 | 33.32876 | 34.26066 | −0.9319 |
| 140 | 33.26371 | 33.95 | −0.68629 |
| 141 | 33.26531 | 33.58823 | −0.32292 |
| 142 | 33.3287 | 33.2 | 0.128699 |
| 143 | 33.44272 | 32.81177 | 0.630953 |
| 144 | 33.5913 | 32.45 | 1.141303 |
| 145 | 33.75531 | 32.13934 | 1.615971 |
| 146 | 33.91479 | 31.90096 | 2.013824 |
| 147 | 34.0512 | 31.75111 | 2.300086 |
| 148 | 34.14954 | 31.7 | 2.449536 |
| 149 | 34.2 | 31.75111 | 2.448889 |
| 150 | 34.19908 | 31.90096 | 2.298122 |
| 151 | 34.14995 | 32.13934 | 2.010614 |
| 152 | 34.06208 | 32.45 | 1.612078 |
| 153 | 33.95013 | 32.81177 | 1.138359 |
| 154 | 33.83232 | 33.2 | 0.63232 |
| 155 | 33.72829 | 33.58823 | 0.140064 |
| 156 | 33.65687 | 33.95 | −0.29313 |
| 157 | 33.63384 | 34.26066 | −0.62682 |
| 158 | 33.67008 | 34.49904 | −0.82896 |
| 159 | 33.77019 | 34.64889 | −0.8787 |
| 160 | 33.9318 | 34.7 | −0.7682 |
| 161 | 34.14563 | 34.64889 | −0.50326 |
| 162 | 34.3963 | 34.49904 | −0.10274 |
| 163 | 34.66383 | 34.26066 | 0.403165 |
| 164 | 34.92561 | 33.95 | 0.975607 |
| 165 | 35.15873 | 33.58823 | 1.570497 |
| 166 | 35.3423 | 33.2 | 2.142301 |
| 167 | 35.45965 | 32.81177 | 2.647875 |
| 168 | 35.5 | 32.45 | 3.05 |
| 169 | 35.45965 | 32.13934 | 3.320307 |
| 170 | 35.3423 | 31.90096 | 3.441339 |
| 171 | 35.15873 | 31.75111 | 3.407615 |
| 172 | 34.92561 | 31.7 | 3.225607 |
| 173 | 34.66383 | 31.75111 | 2.912714 |
| 174 | 34.3963 | 31.90096 | 2.495337 |
| 175 | 34.14563 | 32.13934 | 2.006289 |
| 176 | 33.9318 | 32.45 | 1.481801 |
| 177 | 33.77019 | 32.81177 | 0.958419 |
| 178 | 33.67008 | 33.2 | 0.470078 |
| 179 | 33.63384 | 33.58823 | 0.045608 |
| 180 | 33.65687 | 33.95 | −0.29313 |
| 181 | 33.72829 | 34.26066 | −0.53237 |
| 182 | 33.83232 | 34.49904 | −0.66672 |
| 183 | 33.95013 | 34.64889 | −0.69876 |
| 184 | 34.06208 | 34.7 | −0.63792 |
| 185 | 34.14995 | 34.64889 | −0.49893 |
| 186 | 34.19908 | 34.49904 | −0.29995 |
| 187 | 34.2 | 34.26066 | −0.06066 |
| 188 | 34.14954 | 33.95 | 0.199536 |
| 189 | 34.0512 | 33.58823 | 0.462969 |
| 190 | 33.91479 | 33.2 | 0.714786 |
| 191 | 33.75531 | 32.81177 | 0.94354 |
| 192 | 33.5913 | 32.45 | 1.141303 |
| 193 | 33.44272 | 32.13934 | 1.303385 |
| 194 | 33.3287 | 31.90096 | 1.427737 |

As can be seen in FIG. 2, the contralateral side follows a regular pattern with a 24-hour circadian period.

However, the data for the ipsilateral side (FIG. 1) are more complex; however an ultradian rhythm of 19 hours can be observed.

FIG. 3 shows a diagram of the difference in temperatures of the diagrams of FIGS. 1 and 2. From this diagram can be clearly seen that there are two main peaks at 53 hrs and 170 hrs, together with several peaks at 3, 29, 75, 97, 126 and 149 hrs.

FIG. 4 shows a diagram of the difference in normalized temperatures of the diagrams of FIGS. 1 and 2 using a normalizing procedure as described above. The peaks here are—of course—identical with those of FIG. 3. Such a normalized curve may help to further increase the capability of the device for medical treatment decision support, e.g. in that certain drug or therapy programs are started when the curve rises above a certain threshold or by taking into account the steepness of the normalized curve.

A possible medical treatment would best be started at these peaks; depending on the treatment (and on the drug and/or radiotherapy dosis which needs to be applied) one could consider to only use the main peaks or also apply a drug and/or a radio dosis at one or more of the other peaks as well.

It should be noticed that the optimum times as indicated by FIG. 3 (and in accordance with the present invention) differ greatly from most of the peaks of FIG. 1. Actually the first maximum in skin temperature at the ipsilateral side occurs at the same time as the peak on the contralateral side, which indicates that both tissues do show cell division (cancer and healthy cells) and at which high-dose therapy may have to be avoided to avoid serious side effects even though the tumor cells are dividing. By applying the present invention, one is able to take this into account.

The particular combinations of elements and features in the above detailed embodiments are exemplary only; the interchanging and substitution of these teachings with other teachings in this and the patents/applications incorporated by reference are also expressly contemplated. As those skilled in the art will recognize, variations, modifications, and other implementations of what is described herein can occur to those of ordinary skill in the art without departing from the spirit and the scope of the invention as claimed. Accordingly, the above description is given by way of example only and is not intended to be limiting. The invention's scope is defined in the following claims and the equivalents thereto. Furthermore, reference signs used in the description and claims do not limit the scope of the invention as claimed.

1. A device for medical treatment decision support and/or monitoring the status of a patient comprising
a) a first measuring means, which measures at least one first body parameter at the ipsilateral side of a tumor for at least ≧1 measuring cycle,
b) a second measuring means which measures at least one second body parameter at the contralateral side of a tumor for at least ≧1 measuring cycle,
c) a comparison means to compare the data of the first and second measuring means and/or the fitting curves derived from the first and second measuring means.

2. The device according to claim 1, wherein the at least one first and/or second body parameter includes body temperature, core body temperature, skin surface temperature, activity (body or brain), heart rate, melatonin level, triacylglycerol level, cortisol level, blood pressure and interstitial fluid pressure.

3. The device according to claim 1, wherein the number and nature of the second body parameter(s) are identical to the first body parameter (s).

4. The device according to claim 1, whereby the length of the measuring cycle(s) is from ≧7 h to ≦48 h.

5. The device according to claim 1, wherein the device comprises a fitting means which generates at least one first fitting curve from the data of the first measuring means to determine the acrophase, amplitude, mesor and/or period and/or a fitting means which generates at least one second fitting curve from the data of the second measuring means to determine the acrophase, amplitude, mesor and/or period.

6. The device according to claim 1 wherein the device comprises a normalizing means for normalizing the data from the comparison means in order to normalize either the first and/or second body parameter data and/or the difference of the first and second fitting curve and/or data.

7. The device according to claim 1 wherein the comparison means includes a prediction means which predicts the peak(s) in the difference between the data of the first and second measuring means and/or the fitting curves derived from the first and second measuring means especially for determining an optimal and/or suboptimal time for drug treatment and/or other medical treatment steps.

8. The device according to claim 1 wherein the device comprises a drug administering device which comprises a drug release means, which starts a drug release program based upon the comparison means.

9. A method for the controlled release of drugs and/or monitoring the status of a patient, the method comprising the steps of
a) measuring at least one first body parameter of the patient for at least one first body parameter at the ipsilateral side of a tumor for at least ≧1 measuring cycle,
b) measuring at least one second body parameter of the patient for at least one second body parameter at the contralateral side of a tumor for at least ≧1 measuring cycle,
c) comparing the data out of the first and second body parameters in order to optionally start a drug release program and/or determine an optimal and/or suboptimal time for drug treatment and/or other medical treatment steps.

10. A system incorporating a device according to claim 1 and being used in one or more of the following applications:
medical devices for the administering of drugs, for hyperthermia and for radiotherapy,
medical devices for treatment of chronic diseases.
